(19) United States
(12) Patent Application Publication
(10) Pub. No.: US 2025/0143901 A1
(43) Pub. Date: May 8, 2025

(54) HYBRID ENDOPROSTHESIS SYSTEM

(71) Applicant: W. L. Gore & Associates, Inc., Newark, DE (US)

(72) Inventor: Bryan Reep, Newark, DE (US)

(21) Appl. No.: 18/937,765

(22) Filed: Nov. 5, 2024

Related U.S. Application Data
(60) Provisional application No. 63/597,150, filed on Nov. 8, 2023.

(51) Int. Cl. A61F 2/954; A61F 2/06; A61F 2/07

(52) U.S. Cl. CPC A61F 2/954; A61F 2/07; A61F 2002/065; A61F 2002/068; A61F 2230/0067; A61F 2230/0069

(57) ABSTRACT

An endoprosthesis system including branch and fenestration features for perfusing branch vessels, such as the renal, superior mesenteric, and celiac arteries.

### CROSS-REFERENCE TO RELATED APPLICATION

This application claims the benefit of Provisional Application No. 63/597, 150, filed Nov. 8, 2023, which is incorporated herein by reference in its entirety for all purposes.

### BACKGROUND

Aneurysms occur in blood vessels at sites where, due to age, disease or genetic predisposition of the patient, the strength or resilience of the vessel wall is insufficient to prevent ballooning or stretching of the wall as blood passes through. If the aneurysm is left untreated, the blood vessel wall may expand and rupture, often resulting in death.

To prevent rupturing of an aneurysm, a stent graft may be percutaneously introduced into a blood vessel and deployed to span the aneurysmal sac. Stent grafts include a graft fabric secured to a cylindrical scaffolding or framework of one or more stents. The stent(s) provide rigidity and structure to hold the graft open in a tubular configuration as well as the outward radial force needed to create a seal between the graft and a healthy portion of the vessel wall and provide migration resistance. Blood flowing through the vessel can be channeled through the luminal surface of the stent graft to reduce, if not eliminate, the stress on the vessel wall at the location of the aneurysmal sac. Stent grafts may reduce the risk of rupture of the blood vessel wall at the aneurysmal site and allow blood to flow through the vessel without interruption.

However, various endovascular repair procedures such as the exclusion of an aneurysm require a stent graft to be implanted adjacent to a vascular bifurcation. Often the aneurysm extends into the bifurcation requiring the stent graft to be placed into the bifurcation. A bifurcated stent graft is therefore required in these cases. Modular stent grafts, having a separate main body and branch endoprosthesis are often preferred in these procedures due to the ease and accuracy of deployment. See U.S. Patent Application No. 2008/0114446 to Hartley et al. for an example of a modular stent graft having separate main body and branch stent endoprostheses. In the Hartley et al. publication the main body stent has a fenestration in the side wall that is tailored to engage and secure the side branch stent. The side branch stent in such a configuration is in a “line to line” interference fit with the main body fenestration, causing a potential compromise to the fatigue resistance of the stent-to-stent junction. U.S. Pat. No. 6,645,242 to Quinn presents a more robust stent-to-stent joining configuration. In the Quinn patent, a tubular support, internal to the main body stent, is incorporated to enhance the reliability of the stent to stent joining. U.S. Pat. No. 10,653,540 provides a highly conformable stent graft with a portal for a side branch device.

### SUMMARY

Various examples discussed and shown in this patent specification relate to an endoprosthesis system including bifurcation features and branch features for perfusing branch vessels, such as the renal arteries, superior mesenteric artery, celiac artery, as well as the abdominal aortic bifurcation at the iliac arteries, as well as others.

The various examples provided in this description are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an anterior view of an endoprosthesis system, according to some embodiments.

FIG. 2 is another anterior view of an endoprosthesis system, according to some embodiments.

FIG. 3 is an anterior view of a main body of the system of FIG. 1, according to some embodiments.

FIG. 4 is an anterior view of a contralateral gate extension of the system of FIG. 1, according to some embodiments.

FIG. 5 is an anterior view of main body and contralateral gate extension of the system of FIG. 1, according to some embodiments.

FIG. 6 is a lateral view of the main body of the system of FIG. 1, according to some embodiments.

FIG. 7 is an anterior view of a proximal cuff of the system of FIG. 1, according to some embodiments.

FIG. 8 is an anterior view of main body and proximal cuff components of the system of FIG. 1, according to some embodiments.

FIG. 9 is an anterior view of the main body and another design for the proximal cuff of the system of FIG. 1, according to some embodiments.

FIGS. 10 and 11 show different degrees of overlap of a contralateral limb with a contralateral gate extension of the system of FIG. 1, according to some embodiments.

FIG. 12 shows a delivery system for the system of FIG. 1, according to some embodiments.

FIGS. 13 to 16 show deployment steps for the main body of the system of FIG. 1, according to some embodiments.

FIGS. 17 to 20 show alternative main body configurations for the system of FIG. 1, according to some embodiments.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

“Ipsilateral” and “contralateral” as used in the application are meant to denote relative positions. Generally, “ipsilateral” means one side, or a first side, and “contralateral” means the opposite side, or a second side.

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

### Overview

The various designs addressed in this patent specification relate to an endoprosthesis system 100 (“system”) including a main body endoprosthesis 102, a plurality of side branch endoprostheses 104, including a first side branch endoprosthesis 104a, a second side branch endoprosthesis 104b, and a cuff branch endoprosthesis 104c, although greater or fewer branch endoprostheses are contemplated. The system 100 may also include a superior, or proximal cuff endoprosthesis 106 coupled at a superior, or proximal position to the main body endoprosthesis 102. As illustrated in FIG. 2, the system 100 may also include a contralateral limb endoprosthesis 112 coupled at an inferior position to the contralateral gate extension 110. Additional endoprosthesis components (e.g., superior and/or inferior endoprostheses extending the overall treatment area of the system 100) are contemplated. In general terms, the system 100 acts to carry the blood flow to relieve pressure on damaged, weakened or diseased portions of the vasculature, such as the aorta. In various examples, the system 100 is used for treating an aortic aneurysm, where the system 100 is configured to help direct blood through the aorta and branch vessels associated with the aorta (e.g., renal(s), mesenteric, celiac, iliac(s), and/or hypogastric(s)) while protecting the walls of the aorta.

The system 100 is configured to be endovascularly implanted in (e.g., using a catheter delivery system), and to treat, the abdominal aorta. As described in greater detail below, in various examples, the main body endoprosthesis 102 is configured to be positioned and/or anchored in the abdominal aorta at a location that is superior of, or proximate to, the location above where first and second renal arteries (RA) branch from the aorta, and includes branch features for permitting perfusion of those renal arteries. The system 100 may be anchored in the abdominal aorta at a location that is superior of a location of renal arteries of the patient such that a plurality of side branches are anchorable in renal arteries of the patient for perfusion thereof. In some examples, the main body endoprosthesis 102 also extends above and over the superior mesenteric artery and, optionally the celiac trunk. In some related embodiments, the main body endoprosthesis 102 extends over the superior mesenteric artery and the superior cuff 106 extends over and superior to the celiac trunk. As will be described in greater detail, the main body endoprosthesis 102 and/or superior cuff 105 is positioned and/or anchored at a location superior, or proximal, to the superior mesenteric artery and/or celiac trunk and includes perfusion features for permitting perfusion of one or both of those aortic branch vessels following implantation of the system 100.

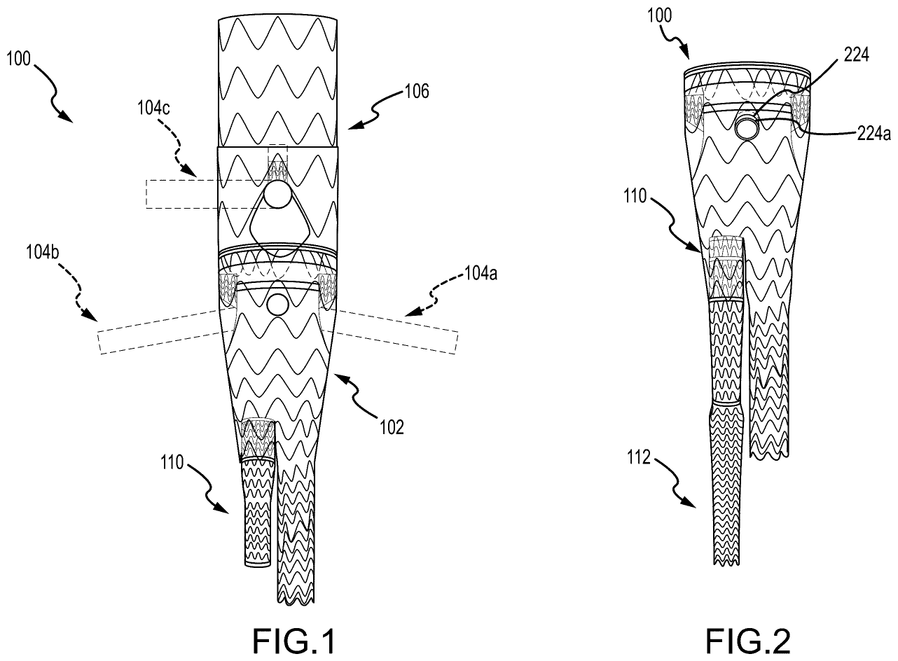

The system 100 may include further stent graft components coupled to the main body endoprosthesis 102 as part of a modular, stent graft solution (e.g., with additional stent graft components secured to the proximal and/or distal end thereof to extend the overall treatment length in a superior direction within the aorta or an inferior direction (e.g., into the common iliac arteries) within the aorta or one or more inferior branches therefrom. For example, as shown in FIG. 1, the system 100 can further include a contralateral gate extension 110. The contralateral gate extension 110 may be a stent graft configured to be telescopically coupled with the contralateral gate of the main body endoprosthesis 102, and to extend toward and into one of the iliac arteries (e.g., when, as appropriate, coupled to a contralateral limb endoprosthesis), with the other, longer leg of the main body endoprosthesis 102 (e.g., also described as an ipsilateral leg) extending toward and/or into the other of the iliac arteries. The contralateral gate extension 110 may be configured to be received in and sealed to an interior gate (e.g., interior contralateral gate 204) such that blood is directed from the main body endoprosthesis 102 into the contralateral gate extension 110.

Each of the side branch endoprostheses 104 are secured to the main body endoprosthesis 102 at one of a plurality of portal features positioned intermediate the proximal and distal ends of the main body endoprosthesis 102. In various examples, the side branches 104 serve to perfuse branch vessels that might otherwise be blocked by implantation of the system 100 to treat an aneurysm. For example, the side branch endoprostheses 104 may be configured to extend from the main body 102 into the renal arteries to help facilitate ongoing perfusion of the kidneys and/or other organs. As described in greater detail below, in various examples, the main body 102 (including referenced as 102a, 102b), is configured to be anchored or otherwise extend in a superior, or proximal direction beyond the renal arteries and includes branch features for permitting perfusion thereof.

The main body 102, side branches 104, and other components (e.g., contralateral gate extension 110 and cuff endoprosthesis 106) are each formed of a graft component supported by a stent, or frame component. Various materials and methods of manufacture are contemplated for those graft and frame components, including the examples subsequently described.

### Main Body Designs

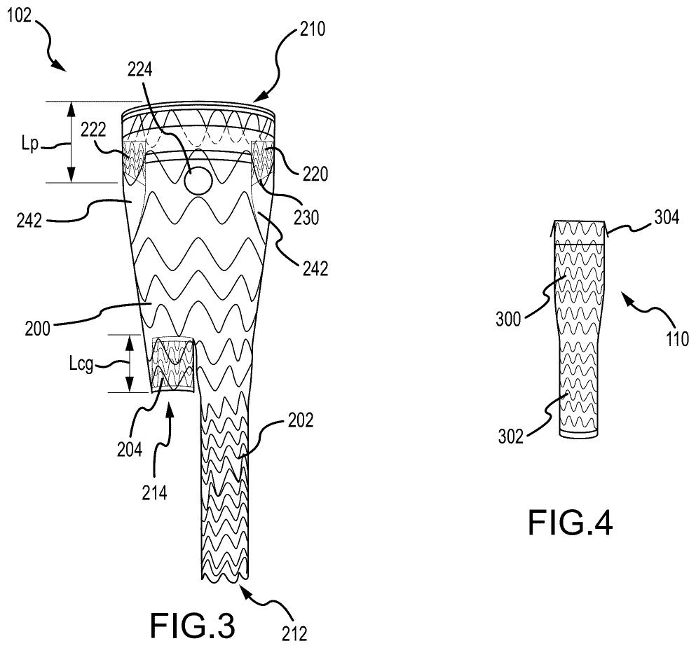

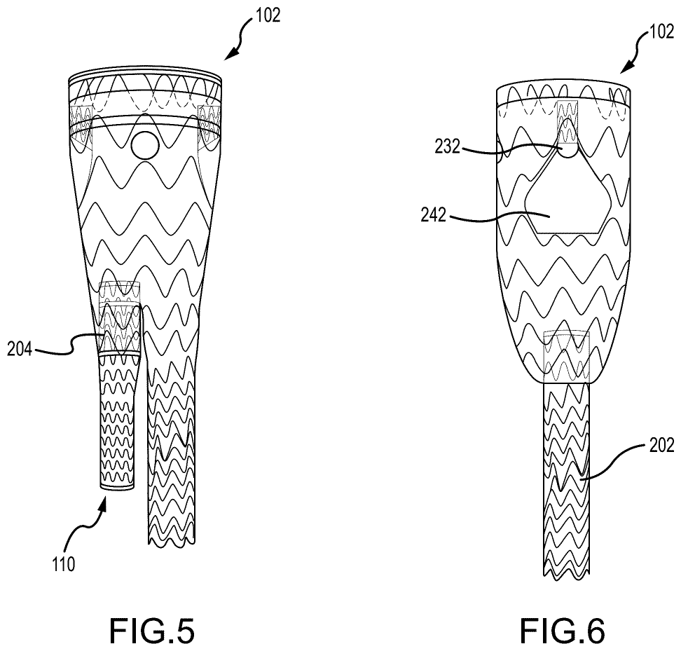

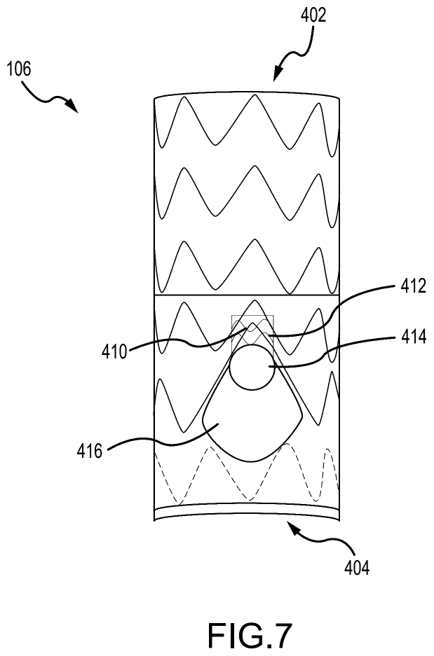

FIG. 3 is an isolated, anterior view of the main body endoprosthesis 102. Generally, the main body endoprosthesis 102 is configured to assume a lower diametric profile, delivery configuration and a larger diametric profile, deployed configuration in the vasculature. As shown, the main body endoprosthesis 102 includes a trunk 200, an ipsilateral leg 202, and an interior contralateral gate 204. In particular, the main body endoprosthesis 102 has an internal lumen (e.g., a bifurcated internal lumen), with a larger proximal inlet 210 corresponding to the trunk 200, a first distal outlet 212 corresponding to the ipsilateral leg 202, and a second distal outlet 214 corresponding to the interior contralateral gate 204. The interior contralateral gate 204 is formed as an internal tubular member that extends generally longitudinally within the primary lumen of the trunk from the second distal outlet 214. In various implementations, the interior contralateral gate is supported by a framework or frame members (e.g., a stent component) such as those described herein. The trunk 200 may include tissue anchors proximate the proximal inlet 210 for gripping vascular tissue of the aorta to reduce the risk of migration from an anchor position.

The trunk 200 includes a first portal 220, a second portal 222, and an anterior fenestration 224. The first and second portals 220, 222 are configured as bilateral internal, renal portals. The design and manufacture of the portals 220, 222 may take a variety of forms, but generally includes an internal tubular member that extends generally longitudinally within the primary lumen of the trunk 200 and terminates at an aperture opening exterior to the trunk 200 through a sidewall of the trunk 220. The internal tubes generally define portals, or gates, into which side branches may be affixed to direct flow from the main body endoprosthesis 102 to one or more branch vessels, such as the renal arteries. Although two internal tubes are shown, any number are contemplated and the current embodiment shown is provided by way example. Because the various portals are otherwise covered features that are hidden from view, the portals are generally shown with thinner lines for purposes of visualization of those hidden features.

For example, as shown, the first portal 220 includes an internal tube 230 into which the first side branch endoprosthesis 104a may be secured, the internal tube 230 extending between a proximal opening and an external opening 232 (FIG. 6) in the sidewall of the main body 202. The second portal 222 is similarly configured, and thus can be described cumulatively to the first portal 220 where each of the portals 220, 222 includes an internal tube with an inlet or origin located within the main lumen of the main body endoprosthesis 102 and an outlet or termination at an external opening in the outer surface of the main body endoprosthesis 102. Although similarly configured in the example of FIG. 1, differing configurations (including greater or fewer portals) are contemplated.

The internal tubes (e.g., internal tube 230) generally extend in a longitudinal direction of the main lumen of the main body endoprosthesis 102. The internal tubes (also described as branch tubes) are optionally made by adding additional graft material that is formed into a tube and coupled (e.g., sewn and/or adhered) to the internal side or external side of the main body endoprosthesis 102 (usually the graft component of the main body endoprosthesis 102) such that the tube is in communication with the main lumen of the main body endoprosthesis 102. In other words, the internal tube 230 can be supported by internal and/or external portions of the main body 12. In various examples, each of the internal tubes are optionally supported by a framework, or stent component. In other examples, the internal tubes are unsupported by additional stent/frame members coupled to the internal tubes. The internal tubes are sized to engage and secure one of the side branches 104 (represented generally by broken lines in FIG. 1) such that they may protrude from the external openings of the first and second portals 220, 222 into a side branch vessel.

As referenced, the origins of the portals 220, 222 are oriented toward the proximal inlet 200 and are generally longitudinally aligned with the main lumen of the main body endoprosthesis 102. In turn, the external openings each extend substantially transverse, or perpendicular to the main lumen, or at some other non-longitudinal angle. The external openings are optionally flush with the outer surface of the main body endoprosthesis 102. The external openings of the portals 220, 222 may be framed, or bounded by one or more stent or frame components (e.g., one of the undulations, or apices may help define an inferior edge and/or the sides of the external openings) associated with the main body endoprosthesis 102.

Although some frame/stent support is contemplated for the external openings, as indicated by the shaded areas in FIG. 3, there may be a first unsupported region 240 located inferior of, or distal to, the external opening 232 of the first portal 220 and a second unsupported region 242 located inferior of, or distal to, the external opening of the second portal 222. Because the unsupported regions 242 are hidden in various views, they are called out and shown with thinner lines in the figures for ease of visualization.

These unsupported regions 240, 242, are generally free from support by any frame component, or stent component, and thus exhibit greater pliability or compliance. In at least this manner, the first and second side branch endoprostheses 104a, 104b may be extended from the external openings of the first and second portals 220, 222 and press against (e.g., deform to some degree) the unsupported regions 240, 242, thus permitting additional flexibility in positioning the extension of the first and second side branch endoprostheses 104a, 104b from the first and second portals 220, 222. Though not shown as being recessed, or otherwise forming depressions, in some examples these areas can be recessed or form pockets relative to the surrounding graft surface of the main body endoprosthesis 102. The unsupported regions 240, 242 may be laterally bounded by diverging portions of a frame component supporting the main body endoprosthesis as shown. The unsupported regions 240, 242 are recessed relative to surrounding (e.g., supported) portions of the main body endoprosthesis 102 to receive one or more portions of branch endoprostheses (e.g., to facilitate deployment thereof).

As shown in FIG. 3, the first and second portals 220, 222 are positioned at a relative angular position from one another, or clocked at a location around the inner circumference of the main body endoprosthesis 102. The first and second portals 220, 222 may be clocked to the medial-lateral axis or offset therefrom. In such an example, the first and second portals 220, 222 are circumferentially offset by approximately 180 degrees from one another +/−20 degrees.

In some examples, the origins define an inset from the proximal inlet 200, or proximal end (or, superior end), of the main body endoprosthesis 102 corresponding to an offset distance Lp defined between the proximal inlet 200 and the center of the external openings of the portals 220, 222. In some examples, the offset distance Lp is 15 mm, for example, although a variety of offset distances are contemplated. The external openings can define a diameter of approximately 6 mm, for example, although a variety of dimensions are contemplated.

In addition (e.g., as a supplement to or as an alternative to the constructs described above), suitable examples of internal tube constructs and portal designs more generally may also be found in U.S. Pat. No. 10,653,540 to Hagaman et al. and U.S. Pat. No. 6,645,242 to Quinn.

As shown in FIG. 3, the main body endoprosthesis 102 includes anterior fenestration 224, although a greater number of fenestrations may also be possible according to some embodiments. The anterior fenestration 224 is positioned between two or more stent, or framework rings, or turns. The anterior fenestration 224 may be pre-fenestrated, or fenestratable. In other words, the graft component of the main body endoprosthesis 102 may be removed with a void (pre-fenestrated) or the graft component may be configured to be removable to form a void (e.g., ablated, cut, punctured or otherwise opened ex vivo or in vivo to form the anterior fenestration 224). The anterior fenestration 224 may include a frame member support (e.g., a ring of material similar to a stent member) and/or may be radiopaque as desired. The anterior fenestration 224 may be oriented to align to a superior mesenteric artery of a patient, for example, although a variety of orientations may be possible. Although, in some embodiments, anchoring the main body endoprosthesis 102 in the aorta includes aligning the anterior fenestration 224 to the superior mesenteric artery of the aorta to perfuse the superior mesenteric artery.

The anterior fenestration 224 or other branching features (e.g., portals 220, 222) may be placed in a general non-patient-specific location or may be placed in patient-specific (e.g., custom) location corresponding to a specific patient anatomy, or generally corresponding to a group of patients' anatomies (e.g., a group having similarly located and sized vessels and branch vessels at a desired treatment site). The anterior fenestration 224 optionally includes a port lining 224a (FIG. 2) received in, and coupled within a boundary of the aperture. Generally, the port lining may be formed as cylindrical tube of material, or is otherwise formed as a tubular member. The port lining may be formed of any of a variety of materials, including any of the graft materials described herein. As shown, the port lining 224a main body endoprosthesis 102, defining an inner portion on an interior side and an outer portion on an outer side of the main body endoprosthesis 102. Although the port lining 224a may have an inner portion and an outer portion, in some embodiments the port lining 224a only extends from the outer surface (only has an outer portion) or the port lining 224a only extends from the inner surface (only has an inner portion). The port lining 224a may be flexible, and formed as a right cylinder (e.g., being free to flex or bend, but not be formed with a bend or curve). If desired, the port lining 224a may have one or more portions that are more resilient, or resistant to flexing, than other portions and/or the port lining 224a may have pre-formed bend(s), taper(s), or other shape.

In some embodiments, the port lining 224a has an overall length of between 1 and 10 mm, such as approximately 5 mm, although a variety of lengths are contemplated. Although port lining is shown in association with each of the apertures of the various fenestration features 300, it should be understood that is not necessarily the case in all embodiments, and that fenestration features without the port lining 224a are also contemplated. Additionally or alternatively, various reinforcements may be provided to reinforce the fenestration features (e.g., the anterior fenestration 224), including one or more of the following: polymeric ring elements, fiber elements, wire elements, stent elements (expandable or self-expanding), and combinations thereof. In different terms, the port lining 224a may be free of any structural reinforcement members (e.g., stents, rings, or other reinforcement) or may be structurally reinforced by one or more structural reinforcement members. In some embodiments, the port lining 224a is reinforced by a collapsible reinforcement element.

Generally, the port lining 224a may assist with any of a variety of functions. For example, the port lining 224a may extend partially into the branch vessel to which the anterior fenestration 224 is aligned. The port lining 224a may act as additional seal material to assist with sealing to the branch vessel. Additionally or alternatively, the port lining 224a may serve as an anchor point and/or sealing feature for use with a branch endoprosthesis. For reference, any of a variety of branch endoprostheses are contemplated, including branch stent-grafts. Examples of suitable branch stent graft include, for example, a suitably configured GORE® VIABAHN® VBX Balloon Expandable Endoprosthesis.

The one or more fenestration features (e.g., the anterior fenestration 224) also optionally include radiopaque markings. For example, a circumferential radiopaque marker may encircle, border, surround, or otherwise be proximate to the anterior fenestration 224. The radiopaque markings may be in the form of a plurality of dots or discrete markings (e.g., four) about the anterior fenestration 224. Additionally, a radiopaque marker in the form of radiopaque material may be impregnated, or otherwise associated with the port lining 224a. Although some examples are provided, any of a variety of radiopaque marker configurations are contemplated, including, for example: a circumferential marker, a dot marker, an impregnated marker, and combinations thereof.

In some examples, the anterior fenestration 224 is unsupported by a frame member, and may be radiopaque (or have edges delineated by radiopaque material) as desired. The anterior fenestration may be arranged to align to a side branch vessel, such as the superior mesenteric artery to facilitate perfusion thereof. In addition, one of the side branch endoprostheses 104 may be delivered through the anterior fenestration 224 into the superior mesenteric artery such that the side branch 104 extends into and anchors within the superior mesenteric artery and couples within the anterior fenestration 224, thereby facilitating perfusion through the anterior fenestration 224 of the superior mesenteric artery. In some examples, the anterior fenestration is clocked to the anterior-posterior axis of the main body endoprosthesis 102. Although not required in various examples, in other examples the anterior fenestration 224 may be configured to be coupled to a side branch component (not shown) or other feature similar to, e.g., cuff branch endoprosthesis 104c (FIG. 1). In some examples, the anterior fenestration 224 is also longitudinally positioned at the same offset distance Lp defined between the proximal inlet 200 and the center of the external openings of the portals 220, 222 (e.g., 15 mm) and may have a diameter of 8 mm, although a variety of dimensions are contemplated.

As shown, the ipsilateral leg 202 extends in an inferior, or distal direction from the trunk 200 a desired length and terminates at the first distal outlet 212. In turn, the contralateral gate 204 does not project substantially from the trunk 200. Because the contralateral gate 204 is everted into the trunk 200 and would otherwise be hidden from view, the contralateral gate 204 is represented by thinner lines in various figures, including FIG. 3. As indicated, the contralateral gate 204 projects or extends proximally, or superiorly, back into the main lumen of the trunk 200. The contralateral gate 204 includes a tubular body having a length and a diameter. The tubular body may be formed of graft component materials (such as those subsequently described) and may be supported by one or more frame components or unsupported as desired. In some examples, the contralateral gate 204 defines a length Lcg of 10 mm, although a variety of dimensions are contemplated. The contralateral gate 204 is configured to receive, be coupled to, and seal to the contralateral gate extension 110 (FIG. 1). In various examples, the contralateral gate 204 is configured to exhibit a minimum overlap of at least 1 cm with the contralateral gate extension 110 to ensure proper sealing/coupling of the two components.

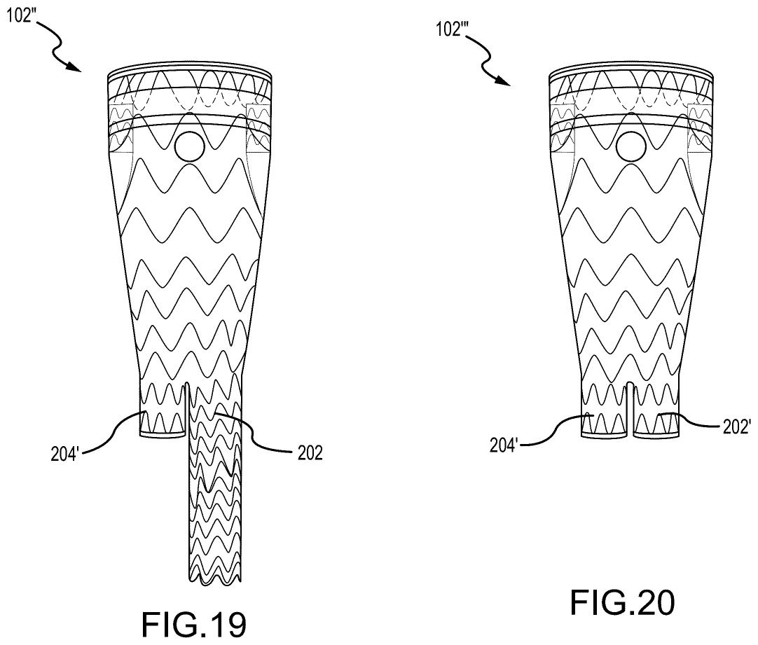

By truncating the contralateral gate 204 (i.e., having at least a portion, or substantially all of the contralateral gate 204 internally disposed with in the trunk 200), the main body endoprosthesis 102 is easier to deliver and deploy into a pre-existing bifurcated endoprosthesis (not shown). For example, a user is not required to accommodate the extension of a stub gate or full-length contralateral leg (compare, e.g., FIGS. 19 and 20) above the bifurcation of a previously implanted main body endoprosthesis. Thus, what are sometimes termed “revision procedures,” may be more easily accomplished with the internal contralateral gate design show in FIG. 3. From this, it will also be apparent that direct implantation at an anatomical bifurcation (i.e., a first time deployment, as opposed to a revision procedure) may be facilitated as well, as, once again, a user is not required to move the main body endoprosthesis 102 as far in a superior direction above the bifurcation in the vasculature in order to avoid interference between the contralateral gate extension/leg and the anatomical bifurcation.

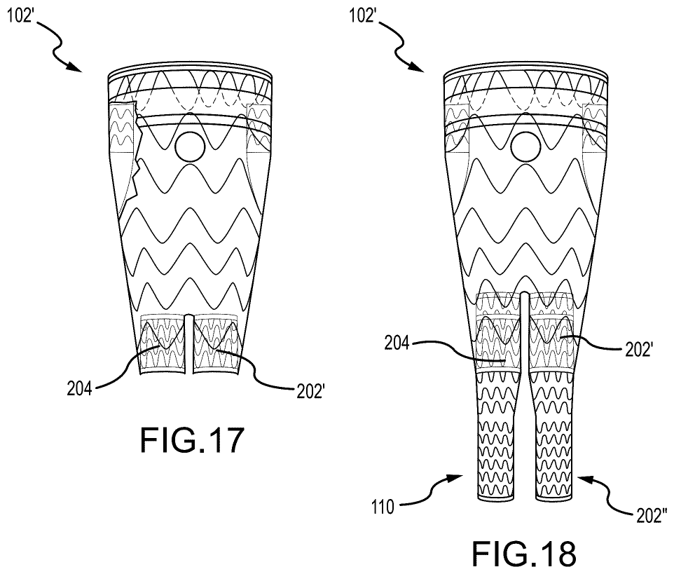

FIGS. 17 and 18 show an alternative design 102′ for the main body endoprosthesis 102 to that shown in FIG. 3. As shown, the main body endoprosthesis 102′ includes the interior contralateral gate 204 with the ipsilateral leg 202 replaced by an interior ipsilateral gate 202′ similar to the contralateral gate 204. As shown in FIG. 18, the interior ipsilateral gate 202′ is a second interior gate that is configured to secure to an ipsilateral gate extension 202″. The ipsilateral gate extension 202″ may be substantially similar in construct and use to the contralateral gate extension 110. The example of FIGS. 17 and 18 may also achieve advantages of more easily being implanted proximate bifurcated structures (whether as part of a revision procedure or implantation directly within the native anatomy). In more direct terms, the provision of such interior gates provides a shorter, overall device for implantation adjacent the bifurcation structure.

FIGS. 19 and 20 show additional alternative designs, respectively, for the main body endoprosthesis 102. Rather than the interior contralateral gate 204, the main body endoprosthesis 102″ of FIG. 19 has a short, external contralateral gate 204′. This configuration is more similar to the configuration of the GORE® EXCLUDER® AAA Endoprosthesis system available from W. L. Gore & Associates, Inc. In turn, FIG. 20 shows a main body endoprosthesis 102″ having two, shorter exterior gates 204′ and 202″. These shorter legs/gates may be combined with leg extensions. Such leg extensions may be similar to the contralateral leg grafts available in association with the GORE® EXCLUDER® AAA Endoprosthesis system available from W. L. Gore & Associates, Inc.

### Branch Designs

The branch endoprostheses 104 may take a variety of forms, including self-expanding and/or balloon expandable stent graft configurations. Generally, the branch endoprostheses 104 are configured to assume a lower diametric profile, delivery configuration and a larger diametric profile, deployed configuration in the vasculature. The branch endoprostheses 104 are generally sized and shaped to be received in one of the internal tubes 150 and extend into a desired side branch vessel, such as the renal arteries. One example of a suitable side branch endoprosthesis corresponds to the GORE® VIABAHN® VBX Balloon Expandable Endoprosthesis available from W. L. Gore & Associates, Inc. Another suitable example, corresponds to the GORE® VIABAHN® Endoprosthesis (self-expanding) available from W. L. Gore & Associates, Inc.

### Gate Extension Designs

The gate extensions described herein, including contralateral gate extension 110, may take a variety of forms, including self-expanding and/or balloon expandable stent graft configurations. The gate extensions generally include one or more graft components supported by one or more frame components. Generally, the gate extensions are configured to assume a lower diametric profile, delivery configuration for delivery into the vasculature and a larger diametric profile, deployed configuration when positioned in the vasculature. For example, the contralateral gate extension 110 is sized and shaped to be received (e.g., telescopically) in the interior contralateral gate 204 and extend into a desired branch vessel, such as the common iliac. The contralateral gate extension 110 may have a generally similar overall shape and construct to that of the GORE® TAG® Thoracic Branch Endoprosthesis available from W. L. Gore & Associates, Inc.

As shown in FIG. 4, the contralateral gate extension 110 has a superior, or proximal portion 300, and an inferior, or distal portion 302, along with a superior, or proximal anchor 304. The proximal portion 300 is widened relative to the distal portion 302 and is configured for insertion into the interior contralateral gate 204 and sealing thereto. The proximal anchor 304, also described as a superior anchor, may assist with ensuring that the contralateral gate extension 110 is retained, or secured, in the interior contralateral gate 204 after deployment. The proximal anchor 304 (FIG. 4) may be in the form of proximal flares (e.g., exposed stent apices that flare outwardly) that may be used to “lock” to the contralateral gate 204. Various dimensions are contemplated for the gate extensions, including the contralateral gate extension 110. In some examples, the proximal, widened portion 300 has a 16 mm diameter and a 15 mm length, the distal, narrowed portion has a 13 mm diameter and a 30 mm length, and the contralateral gate extension 110 has an overall length of 4.5 cm, although a variety of dimensions are contemplated.

### Proximal Cuff Designs

The proximal cuff endoprosthesis 106 may take a variety of forms, including self-expanding and/or balloon expandable stent graft configurations. The proximal cuff endoprosthesis 106 generally includes one or more graft components supported by one or more frame components. Generally, the proximal cuff endoprosthesis 106 is configured to assume a lower diametric profile, delivery configuration during delivery into the vasculature and a larger diametric profile, deployed configuration once positioned in the vasculature.

The proximal cuff endoprosthesis 106 has a proximal inlet 402, a distal outlet 404, and a cuff portal 410. The cuff portal 410 may be similarly constructed to the portals of the main body endoprosthesis 102, the cuff portal 410 including an internal tube 412 and an external opening 414. Generally, the cuff portal 410 is configured to receive and seal to cuff branch endoprosthesis 104c for perfusing one or more branch vessels of the aorta, such as the celiac trunk. In other words, the cuff branch endoprosthesis 104c may serve as a third side branch endoprosthesis of a plurality of side branch endoprostheses that is deployable within the cuff portal 410, and from the cuff portal into the one or more branch vessels. The proximal cuff endoprosthesis 106 may also define an unsupported region 416 adjacent (e.g., inferior, or distal to the external opening 414) for accommodating the cuff branch endoprosthesis 104c as it extends from the cuff portal 410. The unsupported regions 416 may be laterally bounded by diverging portions of a frame component supporting the proximal cuff endoprosthesis 106 as shown.

In some examples, the center of the external opening 414 is about 25 mm above bottom of the proximal cuff endoprosthesis 106, or the distal outlet 404, although a variety of dimensions are contemplated. The internal tube 412 may have a length of about 5 mm to 10 mm in length, for example, although a variety of dimensions are contemplated. The external opening 414 and/or the internal tube 412 may have a diameter of about 8 mm, for example, although a variety of dimensions are contemplated. If desired, a radiopaque marker (such as those previously described) may be associated with the cuff portal 410. For example, a marker may be located at the proximal end of the internal tube 412 and/or the external opening 414.

As an alternative to the cuff portal 410, the proximal cuff endoprosthesis 106 may include a cuff fenestration (not shown) that may be aligned with one or more aortic branch vessels, such as the celiac trunk for perfusion thereof with or without use of a side branch endoprosthesis.

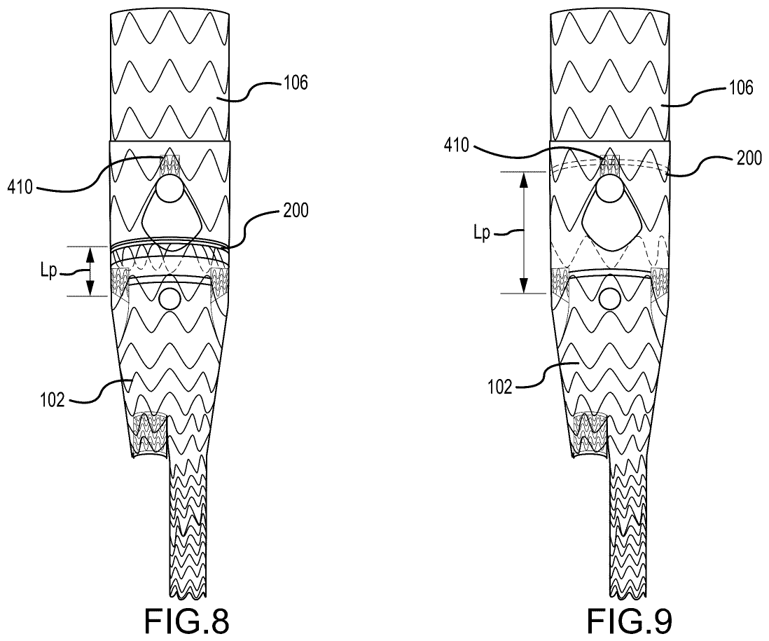

The proximal cuff endoprosthesis 106 is generally sized and shaped to receive (e.g., telescopically) the proximal end of the main body endoprosthesis 102 (e.g., the proximal cuff is implanted prior to the main body), or to be received in the proximal inlet 200 of the main body endoprosthesis 102 (e.g., the proximal cuff is implanted after the main body). In various examples, the proximal cuff endoprosthesis 106 is configured to be implanted prior to the main body endoprosthesis 102 such that the main body endoprosthesis 102 does not interfere with the cuff portal 410 (or cuff fenestration, as applicable). FIG. 8 shows a first arrangement of the proximal cuff endoprosthesis 106 with the main body endoprosthesis 102 received (e.g., telescopically) within the proximal cuff endoprosthesis 106. FIG. 9 shows a second arrangement in which the overall length of the main body endoprosthesis 102 is increased, and in particular the offset distance Lp (e.g., about 15 mm) shown in FIG. 8 is increased as shown in FIG. 9 (e.g., to about 35 mm). As shown in FIG. 9, this arrangement results in an increased overlap (and thus sealing/coupling) between the main body endoprosthesis 102 and the proximal cuff endoprosthesis 106. In some examples, due to the increased overall overlap of the main body endoprosthesis 102 with the celiac trunk, it would be expected that the proximal cuff endoprosthesis 106 would be included in most implementations and implanted prior to the main body endoprosthesis 102 in order to preserve the ability to perfuse the celiac trunk.

### Contralateral Limb

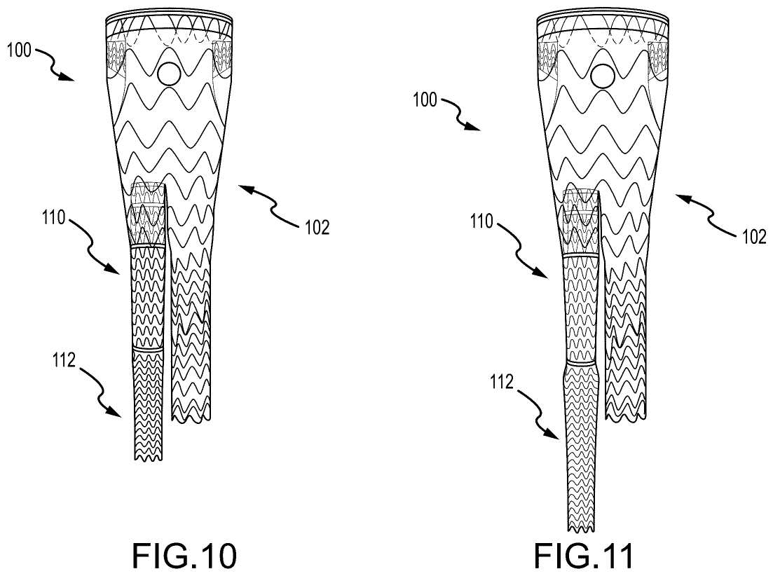

One example of a suitable contralateral limb 110 is the contralateral limb endoprosthesis sold as part of the GORE® EXCLUDER® AAA Endoprosthesis system available from W. L. Gore & Associates, Inc. As illustrated in FIGS. 10 and 11, the contralateral gate extension 110 and interior contralateral gate 204 can be configured to achieve varying degrees of overlap with the contralateral limb endoprosthesis 112 to achieve varying degrees of extension/length. In some examples, the difference between maximum overlap and minimum overlap achievable between the components is about 15 mm, although a variety of dimensions are contemplated.

### Materials

The materials used for the graft components associated with the various endoprostheses can include any material which is suitable for use as a graft in the chosen body lumen. The graft components for the various endoprostheses can be composed of the same or different materials between the various endoprostheses. The graft components can comprise multiple layers of material that can be the same material or different materials. The graft components may have a layer that is formed into a tube (innermost tube) and an outermost layer that is formed into a tube (outermost tube).

Many graft materials are known, particularly known are those that can be used as vascular graft materials. The graft materials can be extruded, coated or formed from wrapped films, or a combination thereof.

Polymers, biodegradable and natural materials can be used for specific applications. Biocompatible materials in particular are contemplated for the various graft components. In certain instances, the graft components may include a fluoropolymer, such as a polytetrafluoroethylene (PTFE) polymer or an expanded polytetrafluoroethylene (ePTFE) polymer. In some instances, the graft components may be formed of, such as, but not limited to, a polyester, a silicone, a urethane, a polyethylene terephthalate, or another biocompatible polymer, or combinations thereof. In some instances, bioresorbable or bioabsorbable materials may be used, for example a bioresorbable or bioabsorbable polymer. In some instances, the graft can include Dacron, polyolefins, carboxy methylcellulose fabrics, polyurethanes, or other woven, non-woven, or film elastomers.

Biocompatible materials may be used for the various frame components (also described as framework, frame members, or stent components, for example), associated with the endoprostheses described herein. For example, nitinol (NiTi) may be used as the material of the frame or stent (and any of the frames discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the frame. The super-elastic properties and softness of NiTi may enhance the conformability of the stent. In addition, NiTi can be shape-set into a desired shape. That is, NiTi can be shape-set so that the frame tends to self-expand into a desired shape when the frame is unconstrained, such as when the frame is deployed out from a delivery system.

### Main Body Delivery System Designs

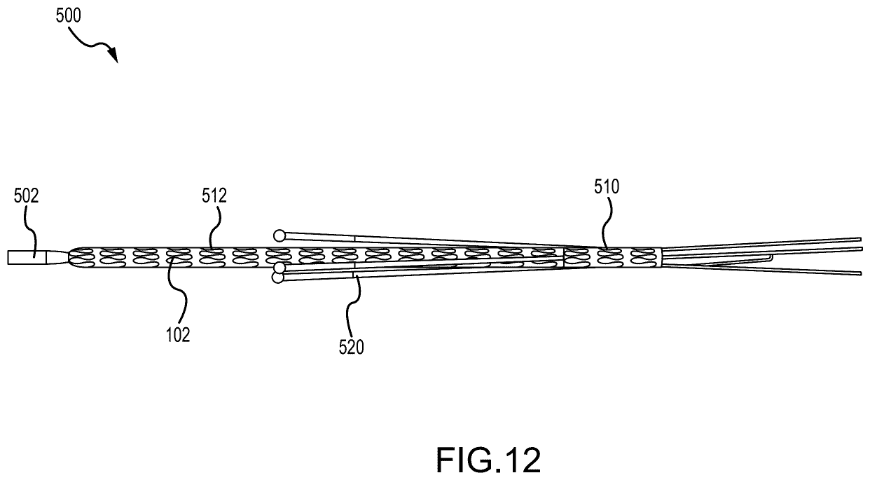

FIG. 12 shows an example delivery system 500 for the main body endoprosthesis 102. As shown, the delivery system 500 includes a delivery catheter 502 carrying the main body endoprosthesis 102, or in different terms upon which the main body endoprosthesis 102 is mounted. The delivery system 500 includes one or more constraints (e.g., retaining sleeves) for maintaining the main body endoprosthesis 102 in a diametrically compacted state on the delivery catheter 502. For example, the delivery system 500 may include a proximal sleeve 510 corresponding to a highlighted length of the main body endoprosthesis 102 (e.g., about 3 cm long, although other dimensions are contemplated) and a main sleeve 512 corresponding to a remainder of the length of the main body endoprosthesis 102 and potentially extending over the proximal sleeve 510. For reference, the proximal sleeve 510 and main sleeve 512 may be translucent, and thus underlying portions of the main body endoprosthesis (e.g., stent elements) may be visible through the proximal sleeve 510 and/or main sleeve 512.

The delivery system 500 is configured to permit sequential release of the proximal and distal sleeves 510, 512. As shown, the delivery system 500 also includes pre-cannulated guidewire tubes 520 placed through the first and second portals 220, 222, and the anterior fenestration 224. The guidewire tubes are optionally used to pre-cannulate the corresponding portal/fenestration with a guidewire for delivery of the branches 104 and/or alignment to the vasculature. Suitable examples of guidewire tubes can be found in U.S. Pat. No. 9,622,886 to Hamer et al., although a variety of configurations are contemplated.

### Metho of Main Body Deployment Overview

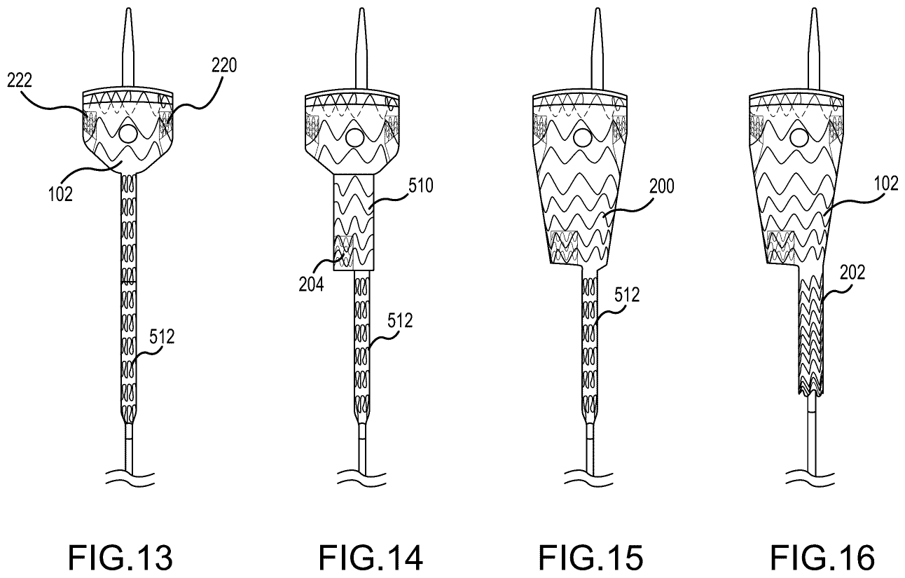

FIGS. 13 through 16 are illustrative of an example delivery sequence of the main body endoprosthesis 102. As shown, the proximal portion of the main body endoprosthesis 102 is permitted to expand (e.g., by partially releasing the main sleeve 512 for initial positioning and anchoring in the vasculature, such as the abdominal aorta). In particular, the proximal end portion extending to just below the portals 220, 222 may be deployed, with the proximal end being fully deployed as desired. A suitable delivery system for use with the main body endoprosthesis 102 may be that associated with the GORE® EXCLUDER® Conformable AAA Endoprosthesis available from W. L. Gore & Associates, Inc. For example, such systems would include the ability to reconstrain the proximal end of the main body endoprosthesis 102.

As shown in FIG. 14, once positioned as desired, the main sleeve 512 may be further released with the proximal sleeve 510 permitting the intermediate portion of the main body endoprosthesis 102 (e.g., between the portals 220, 222 and the contralateral gate) to expand to an intermediate diameter, or partially deployed diameter. In some examples, this feature helps facilitate deployment of the first and second side branch endoprostheses 104a, 104b through the respective portals 220, 222 without undue interference with the main body endoprosthesis 102.

As shown in FIG. 15, the proximal sleeve 510 may then be released fully deploying the intermediate portion of the main body endoprosthesis 102 (e.g., the full length of the trunk 200), including the interior contralateral gate 204. At this point, the contralateral gate extension 110 (not shown) may be delivered into the contralateral gate 204 (e.g., via an associated delivery catheter) and deployed therein. As shown, the ipsilateral leg 202 remains constrained permitting better control of the main body endoprosthesis 102 as well as helping to ensure that the ipsilateral leg 202 does not interfere with contralateral gate extension 110 deployment. In some embodiments, deploying the contralateral gate extension 110 through the interior contralateral gate 204 includes securing the contralateral gate extension 110 to the interior contralateral gate 204 using the superior anchor.

As shown in FIG. 16, the remainder of the main sleeve 512 may be released allowing expansion of the ipsilateral leg 202 and full deployment of the main body endoprosthesis 102. Although this example sequence has been provided, it should be understood that a variety of deployment sequences and associated delivery systems are contemplated.

### Notice

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. An endoprosthesis system for treating an aortic aneurysm by helping direct blood through an aorta of a patient while protecting the walls of the aorta from further pressure from the blood flow, the endoprosthesis system comprising:
a main body endoprosthesis having a proximal inlet, a first distal outlet, and a second distal outlet, the main body including a trunk defining the proximal inlet and an interior gate positioned within the trunk and defining one of the first and second distal outlets, the trunk including a first portal, a second portal, and an anterior fenestration;
a plurality of side branch endoprostheses, including a first side branch endoprosthesis and a second side branch endoprosthesis, each configured to be received in one of the first and second portals and to extend from first and second portals; and
a contralateral gate extension configured to be received in and sealed to the interior gate such that blood is directed from the main body into the contralateral gate extension;
wherein the system is configured to be endovascularly implanted in, and to treat the abdominal aorta, the main body endoprosthesis being configured to be anchored in the abdominal aorta at a location that is superior of a location of renal arteries of the patient such that the plurality of side branch endoprostheses are anchorable in renal arteries of the patient for perfusion thereof.

2. The system of claim 1, wherein the interior gate is formed as an internal tubular member that extends generally longitudinally within the primary lumen of the trunk.

3. The system of claim 1, wherein the interior gate is supported by a frame component.

4. The system of claim 1, wherein the anterior fenestration is configured to align to a superior mesenteric artery of a patient.

5. The system of claim 1, wherein the first and second portals are circumferentially offset by approximately 180 degrees.

6. The system of claim 1, wherein the anterior fenestration is circumferentially offset from each of the first and second portals by about 90 degrees.

7. The system of claim 1, further comprising a proximal cuff endoprosthesis having a cuff portal configured to receive a third side branch endoprosthesis of the plurality of side branch endoprostheses, the proximal cuff being configured to receive the main body endoprosthesis, the cuff portal being configured to perfuse the celiac trunk through the third side branch endoprosthesis.

8. The system of claim 1, further comprising an ipsilateral gate extension, wherein the main body endoprosthesis includes a second interior gate within the trunk that defines one of the first and second distal outlets, the second interior gate configured to receive the ipsilateral gate extension.

9. The system of claim 1, wherein the trunk of the main body endoprosthesis defines a first unsupported region adjacent and distal to the first portal, the first unsupported region being free of any frame component.

10. The system of claim 9, wherein the main body endoprosthesis includes a frame component and the unsupported region is laterally bounded by diverging portions of a frame component.

11. The system of claim 9, wherein the unsupported region is recessed relative to surrounding portions of the main body endoprosthesis.

12. A method of deploying a main body endoprosthesis of an endoprosthesis system for treating an aortic aneurysm configured by helping direct blood through an aorta of a patient while protecting the walls of the aorta from further pressure from the blood flow, the method comprising:
positioning the main body endoprosthesis in the aorta of the patient using a delivery system, the delivery system including a catheter, a proximal sleeve, and a main sleeve;
deploying a proximal end of the main body endoprosthesis to anchor the main body endoprosthesis in the aorta by partially releasing the main sleeve;
deploying an intermediate portion of the main body endoprosthesis to an intermediate diameter, the proximal sleeve maintaining the intermediate portion at the intermediate diameter;
deploying first and second branch endoprostheses through first and second portals of the main body endoprosthesis while the intermediate portion is at the intermediate diameter to perfuse first and second renal arteries of a patient;
fully deploying the intermediate portion of the main body endoprosthesis by releasing the proximal sleeve;
deploying a contralateral gate extension through an interior contralateral gate positioned in a trunk of the main body endoprosthesis; and
deploying an ipsilateral leg of the main body endoprosthesis by further releasing the main sleeve.

13. The method of claim 12, further comprising deploying a proximal cuff prior to deploying the proximal end of the main body, wherein deploying the proximal end of the main body includes deploying the proximal end of the main body in the proximal cuff.

14. The method of claim 13, wherein the proximal cuff has a cuff portal, the method further comprising deploying a branch endoprosthesis through the cuff portal and extending the branch endoprosthesis into a celiac trunk of the patient to perfuse the celiac trunk.

15. The method of claim 12, wherein the main body endoprosthesis further includes an anterior fenestration and further wherein anchoring the main body endoprosthesis in the aorta includes aligning the anterior fenestration to the superior mesenteric artery (SMA) of the aorta to perfuse the SMA.

16. The method of claim 12, wherein the contralateral gate extension includes a proximal anchor, and wherein deploying the contralateral gate extension through the interior contralateral gate includes securing the contralateral gate extension to the interior contralateral gate using the proximal anchor.

17. The method of claim 12, wherein the interior gate is formed as an internal tubular member that extends generally longitudinally within the primary lumen of the trunk.

18. The method of claim 12, wherein the interior gate is supported by a frame component.

19. The method of claim 12, wherein the trunk of the main body endoprosthesis defines a first unsupported region adjacent and distal to the first portal, the first unsupported region being free of any frame component, the first branch endoprosthesis passing adjacent the first unsupported region.

20. The system of claim 12, wherein the main body endoprosthesis includes a frame component and the unsupported region is laterally bounded by diverging portions of a frame component.

21. The system of claim 12, wherein the unsupported region is recessed relative to surrounding portions of the main body endoprosthesis and the first branch endoprosthesis is received in the recess.
